(19) United States
(12) Patent Application Publication
(10) Pub. No.: US 2024/0358483 A1
(43) Pub. Date: Oct. 31, 2024

(54) FLEXIBLE INTELLIGENT MANUFACTURING AND PRODUCTION METHOD AND SYSTEM FOR CUSTOMIZED DENTURE

(71) Applicants: BETAMARS DENTALLAB CO., LTD., Zhuhai (CN); DIGITAL DIRECT DENTAL, Zhuhai (CN)

(72) Inventors: Wenhu LIU, Zhuhai (CN); Tie BIN, Zhuhai (CN); Zhiyuan LIU, Zhuhai (CN); Qiuxia LI, Zhuhai (CN); Guoying XIAO, Zhuhai (CN)

(73) Assignees: BETAMARS DENTALLAB CO., LTD., Zhuhai (CN); DIGITAL DIRECT DENTAL, Zhuhai (CN)

(21) Appl. No.: 18/764,261

(22) Filed: Jul. 4, 2024

Related U.S. Application Data
(63) Continuation of application No. PCT/CN2023/089119, filed on Apr. 19, 2023.

(30) Foreign Application Priority Data
Jun. 22, 2022 (CN) 202210708257.7

(51) Int. Cl. A61C 13/00

(52) U.S. Cl. CPC A61C 13/0004; A61C 13/0007

(57) ABSTRACT

A flexible intelligent manufacturing and production system for a customized denture includes a server, a processing system, a cutting device, a tooth unloading device and a sintering device. The processing system, the cutting device, the tooth unloading device and the sintering device all have a function of receiving/sending commands, and are all connected to the server signal. The processing system is used for processing a three-dimensionality (3D) tooth model file and a 3D zirconium block graphic file, and generating a file. The server is used for receiving/sending the file; the cutting device is used for receiving a tool path file and cutting a zirconium block according to the tool path file. The tooth unloading device is used for receiving/sending a coordinate file and cutting a connecting rod according to the coordinate file. The sintering device is used for sintering a denture.

### CROSS-REFERENCE TO RELATED APPLICATIONS

This application is a continuation of International Application No. PCT/CN2023/089119, filed on Apr. 19, 2023, which claims priority to Chinese Patent Application No. 202210708257.7, filed on Jun. 22, 2022. All of the aforementioned applications are incorporated herein by reference in their entireties.

### TECHNICAL FIELD

The present disclosure herein relates to the technical field of denture processing, and in particular to a flexible intelligent manufacturing and production method and a flexible intelligent manufacturing and production system for a customized denture.

### BACKGROUND

Denture processing refers to the production and processing of oral denture materials in the medical device industry to provide patients with artificial dental restorations. However, in the current processing process of the denture automated production line, it is impossible to track each tooth at a certain station. For the specific location, it takes a lot of time to find the corresponding teeth during the production process, and during the calibration process, the teeth are embedded into the dental mold for calibration. However, it requires experienced technicians to make judgments in the inspection process, which not only consumes a lot of manpower, but also inevitably leads to errors, omissions, and losses during the inspection.

### SUMMARY

The technical problem to be solved by the present disclosure is to overcome the shortcomings of the prior art. The first object of the present disclosure is to provide a flexible intelligent manufacturing and production method for a customized denture that improves production efficiency and locates the position of each tooth.

The second object of the present disclosure is to provide a flexible intelligent manufacturing and production system for a customized denture that improves production efficiency and locates the position of each tooth.

The technical solution adopted by the present disclosure is: the flexible intelligent manufacturing and production method for the customized denture includes the following steps:
designing a 3D tooth model file, and uploading the 3D tooth model file to a server;
retrieving the 3D tooth model file to a 3D zirconium block graphic file for typesetting, and generating a tool path file;
numbering each tooth according to a typeset graphic file, setting coordinates of each tooth, and generating a coordinate file;
uploading the tool path file and the coordinate file to the server, the server transmitting the coordinate file to a tooth unloading device and transmitting the tool path file to a cutting device, and the cutting device grabbing a corresponding zirconium block for cutting;
transporting a cut zirconium block to a tooth unloading station, the tooth unloading device moving, according to the coordinate file, the zirconium block to a corresponding coordinate position for cutting operation of a connecting rod, taking out a cut tooth to a sintering device, and the tooth unloading device uploading the coordinate file to the server; and
the sever recording the coordinates of the tooth sintered in the sintering device and transferring the coordinates to a next station for positioning operation, and the sintering device sintering the tooth.

Furthermore, the steps of numbering each tooth according to the typeset graphic file, setting coordinates of each tooth, and generating the coordinate file include:
numbering each tooth according to the typeset graphic file;
the processing system uses a center of the zirconium block as an origin of the coordinate, and a center of each tooth as a positioning coordinate; and
generating the coordinate file based on the positioning coordinate.

Furthermore, the steps of uploading the tool path file and the coordinate file to the server, the server transmitting the coordinate file to a tooth unloading device and transmitting the tool path file to a cutting device, and the cutting device grabbing a corresponding zirconium block for cutting include:
uploading the tool path file and the coordinate file to the server, and the server transmitting the coordinate file to the tooth unloading device and transmitting the tool path file to the cutting device;
the server commands a manipulator of the cutting device to grab the corresponding zirconium block and bring it to the cutting device for cutting; and
after the cutting is completed, the manipulator grabs the cut zirconium block and sends it to a conveyor line.

Furthermore, the steps of transporting a cut zirconium block to a tooth unloading station, the tooth unloading device moving, according to the coordinate file, the zirconium block to a corresponding coordinate position for cutting operation of a connecting rod, taking out a cut tooth to a sintering device, and the tooth unloading device uploading the coordinate file to the server include:
transporting the zirconium block through the conveyor line, and the manipulator of the tooth unloading device grabs the cut zirconium block from the conveyor line and places it at the tooth unloading station;
according to a coordinate value for each tooth determined by the coordinate file sent from the server, the tooth unloading device confirms that the zirconium block moves to the corresponding coordinate position for a tooth unloading personnel to perform the cutting operation of the connecting rod;
the tooth unloading personnel takes out the tooth with the connecting rod cut off and places it at the position of a sintering plate designated by the processing system; and
the tooth unloading device uploads an updated coordinate file to the server.

Furthermore, in the steps of retrieving the 3D tooth model file to the 3D zirconium block graphic file for typesetting, and generating the tool path file, the zirconium block is information-bound to the 3D zirconium block graphic file through a Quick Response (QR) code.

The present disclosure also provides a flexible intelligent manufacturing and production system for a customized denture, wherein the flexible intelligent manufacturing and production system for the customized denture includes a server, a processing system, a cutting device, a tooth unloading device and a sintering device; the processing system, the cutting device, the tooth unloading device and the sintering device all have the function of receiving/sending commands, and are all connected to the server signal; the processing system is used for processing a 3D tooth model file and a 3D zirconium block graphic file, and generating a file; the server is used for receiving/sending the file; the cutting device is used for receiving a tool path file and cutting a zirconium block according to the tool path file; the tooth unloading device is used for receiving/sending a coordinate file and cutting a connecting rod according to the coordinate file; and sintering device is used for sintering a denture.

Furthermore, the flexible intelligent manufacturing and production system for the customized denture further comprises a glazing and sintering device, a sandblasting device, a polishing device and a quality inspection and packaging device; the glazing and sintering device, the sandblasting device, the polishing device and the quality inspection and packaging device all have the function of receiving/sending commands, and are all connected to the server signal; the glazing and sintering device is used for glazing and sintering a denture; the sandblasting device is used for sandblasting dentures, the polishing device is used for polishing the denture; and the quality inspection and packaging device is used for quality inspection and packaging of the denture.

Furthermore, the processing system includes a computer, a CAD design software is installed in the computer, and the 3D tooth model file designed based on the CAD design software is uploaded to a designated folder on the server.

Furthermore, the cutting device includes a first manipulator, a denture engraving machine and a conveyor belt; the denture engraving machine is used for cutting a tooth in the zirconium block; the first manipulator is used for grabbing the corresponding zirconium block to the denture engraving machine for cutting according to the server's command, and placing the cut zirconium block on the conveyor belt; and the conveyor belt is used for transporting the zirconium block to the tooth unloading device.

Furthermore, the tooth unloading device includes a second manipulator, a tooth unloading station, a tooth unloading processing hole and a cutting module; the second manipulator is used for grabbing the zirconium block on the conveyor belt to the tooth unloading station; the tooth unloading station is used for moving the zirconium block to the corresponding coordinate position according to a coordinate value of each tooth, so that the corresponding tooth in the zirconium block is positioned beneath the tooth unloading processing hole; and the cutting module is used by an operator to cut the corresponding tooth from the tooth unloading processing hole.

The beneficial effects of the present disclosure are: compared with the shortcomings of the prior art, in the present disclosure, in cases where the operator does not have the right to take any teeth, all the assignments are completed by the processing system, thereby confirming the exact location of each tooth at a specific station. The processing system tracks the progress to ensure that every station in the entire production process knows the relevant information for each tooth at their station, thereby improving production efficiency, reducing the phenomenon of constantly searching for teeth during the production process, and avoiding the phenomenon of errors, omissions, and losses that occur during the searching process. Therefore, the described flexible intelligent manufacturing and production method for the customized denture has the advantages of improving production efficiency and locating the position of each tooth.

### BRIEF DESCRIPTION OF THE DRAWINGS

FIG. 1 is a flow chart schematic diagram of a flexible intelligent manufacturing and production method for a customized denture provided by an embodiment of the present disclosure.

FIG. 2 is a flow chart schematic diagram of a flexible intelligent manufacturing and production method for a customized denture provided by an embodiment of the present disclosure.

FIG. 3 is a flow chart schematic diagram of a flexible intelligent manufacturing and production method for a customized denture provided by an embodiment of the present disclosure.

FIG. 4 is a flow chart schematic diagram of a flexible intelligent manufacturing and production method for a customized denture provided by an embodiment of the present disclosure.

FIG. 5 is a structure schematic diagram of a flexible intelligent manufacturing and production system for a customized denture provided by an embodiment of the present disclosure.

FIG. 6 is a plan view of a processing system of a flexible intelligent manufacturing and production system for a customized denture provided by an embodiment of the present disclosure.

FIG. 7 is a plan view of a cutting device of a flexible intelligent manufacturing and production system for a customized denture provided by an embodiment of the present disclosure.

FIG. 8 is a plan view of a tooth unloading device of a flexible intelligent manufacturing and production system for a customized denture provided by an embodiment of the present disclosure.

### DETAILED DESCRIPTION OF THE EMBODIMENTS

Embodiments of the present disclosure will be described in more detail below with reference to the accompanying drawings. Although certain embodiments of the present disclosure are shown in the accompanying drawings, it should be understood that the present disclosure can be implemented in various forms and should not be interpreted as being limited to the embodiments described here. On the contrary, these embodiments are provided to gain a more thorough and complete understanding of the present disclosure. It should be understood that the drawings and embodiments of the present disclosure are for illustrative purposes only and are not intended to limit the scope of protection of the present disclosure.

It should be understood that the steps described in the method embodiments of the present disclosure can be executed in different orders, and/or executed in parallel. Furthermore, the method embodiments may include additional steps and/or omit executing the steps shown. The scope of the present disclosure is not limited in this regard.

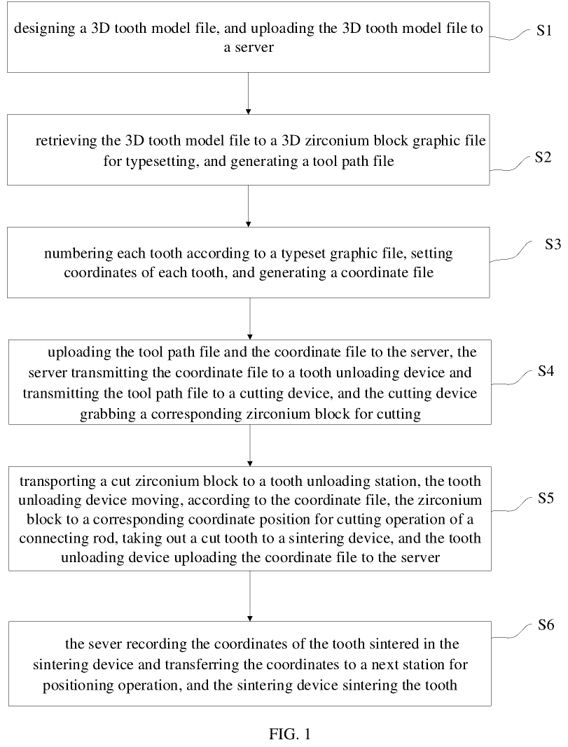

As shown in FIG. 1, the present disclosure provides a flexible intelligent manufacturing and production method for a customized denture. The flexible intelligent manufacturing and production method for the customized denture includes the following steps.
Step S1: designing a three-dimensionality (3D) tooth model file, and uploading the 3D tooth model file to a server.

Specifically, a 3D tooth model file is designed based on computer-aided design (CAD) software, and the 3D tooth model file is uploaded to a designated folder on the server.
Step S2: retrieving the 3D tooth model file to a 3D zirconium block graphic file for typesetting, and generating a tool path file.

Specifically, the 3D zirconium block graphic file in the processing system is retrieved, the 3D tooth model file in the server is retrieved, the 3D tooth model file is retrieved to the 3D zirconium block graphic file for typesetting, and the processing system generates the tool path file. It should be noted that the typesetting personnel retrieve the 3D zirconium block graphic file from the processing system. The zirconium block is provided with a QR code. The zirconium block is information-bound to the 3D zirconium block graphic file through a QR code. The 3D zirconium block graphic file can be an empty 3D zirconium block graphic file, or a 3D zirconium block graphic file containing part of the teeth. In the process of retrieving the 3D tooth model file to a 3D zirconium block graphic file for typesetting, through the operation of the processing system, one or more 3D tooth model files can be retrieved to the 3D zirconium block graphic file for typesetting until the 3D zirconium block graphic file is filled with the 3D tooth model files.
Step S3: numbering each tooth according to a typeset graphic file, setting coordinates of each tooth, and generating a coordinate file.

Specifically, each tooth can be numbered. For instance, if there are 10 teeth on the zirconium block, these 10 teeth can be numbered (1, 2, 3, 4, 5, 6, 7, 8, 9, 10). Taking the center of the zirconium block as the origin, the center of each tooth is clicked through the processing system to set the positioning coordinate, and a coordinate file is generated.
Step S4: uploading the tool path file and the coordinate file to the server, the server transmitting the coordinate file to a tooth unloading device and transmitting the tool path file to a cutting device, and the cutting device grabbing a corresponding zirconium block for cutting.

Specifically, after the coordinate file is determined and uploaded to the server, the server can obtain the coordinates of these teeth in the zirconium block. After the server transmits the tool path file to the cutting device, the cutting device will cut the teeth in the zirconium block based on the tool path file.
Step S5: transporting a cut zirconium block to a tooth unloading station, the tooth unloading device moving, according to the coordinate file, the zirconium block to a corresponding coordinate position for cutting operation of a connecting rod, taking out a cut tooth to a sintering device, and the tooth unloading device uploading the coordinate file to the server.

Specifically, the manipulator of the tooth unloading device grabs the zirconium block on the conveyor line to the tooth unloading station. Based on the coordinate value for each tooth determined by the typesetting personnel and generated by the server, the tooth unloading device confirms the corresponding coordinate position for the zirconium block to be moved to for the tooth unloading to operate. At this point, the corresponding tooth on the zirconium block is positioned beneath the tooth unloading processing hole. The tooth unloading operator then cuts off the connecting rod of the tooth from the tooth unloading processing hole and takes out the tooth, placing it in the designated sintering plate. The order of tooth unloading follows the sequential numbering of the teeth.
Step S6: the sever recording the coordinates of the tooth sintered in the sintering device and transferring the coordinates to a next station for positioning operation, and the sintering device sintering the tooth.

Furthermore, the subsequent operations of glazing and sintering device, sandblasting device, polishing device, and quality inspection and packaging device all use the above method to perform real-time positioning and tracking of the coordinates of the teeth at each station.

It should be noted that during this process, in cases where the operator does not have the right to take any teeth, all the assignments are completed by the processing system, thereby confirming the exact location of each tooth at a specific station. The processing system tracks the progress to ensure that every station in the entire production process knows the relevant information for each tooth at their station, thereby improving production efficiency, reducing the phenomenon of constantly searching for teeth during the production process, and avoiding the phenomenon of errors, omissions, and losses that occur during the searching process. Therefore, the described flexible intelligent manufacturing and production method for the customized denture has the advantages of improving production efficiency and locating the position of each tooth.

In the above Step S3, as shown in FIG. 2, the steps of numbering each tooth according to the typeset graphic file, setting coordinates of each tooth, and generating the coordinate file include the follows.
Step S31: numbering each tooth according to the typeset graphic file.
Step S32: the processing system uses a center of the zirconium block as an origin of the coordinate, and a center of each tooth as a positioning coordinate.
Step S33: generating the coordinate file based on the positioning coordinate.

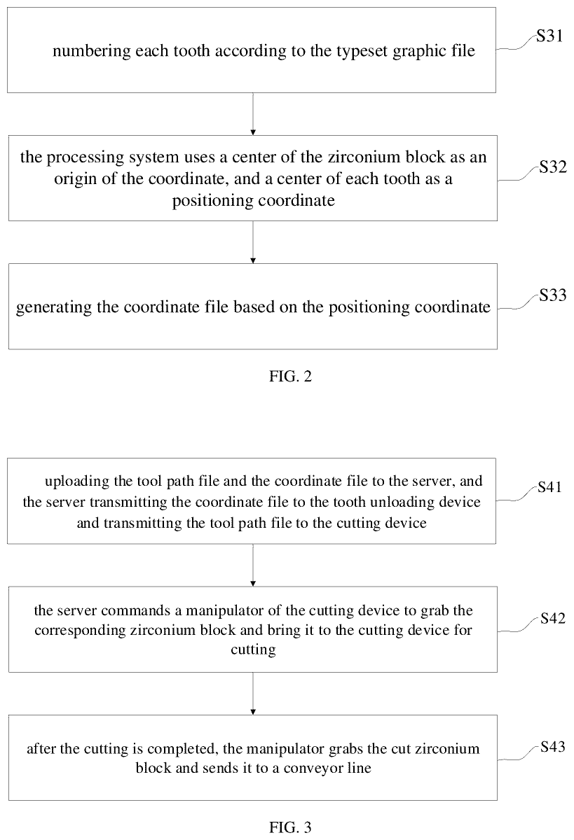

In the above Step S4, as shown in FIG. 3, the steps of uploading the tool path file and the coordinate file to the server, the server transmitting the coordinate file to a tooth unloading device and transmitting the tool path file to a cutting device, and the cutting device grabbing a corresponding zirconium block for cutting include the follows.
Step S41: uploading the tool path file and the coordinate file to the server, and the server transmitting the coordinate file to the tooth unloading device and transmitting the tool path file to the cutting device.
Step S42: the server commands a manipulator of the cutting device to grab the corresponding zirconium block and bring it to the cutting device for cutting.
Step S43: after the cutting is completed, the manipulator grabs the cut zirconium block and sends it to a conveyor line.

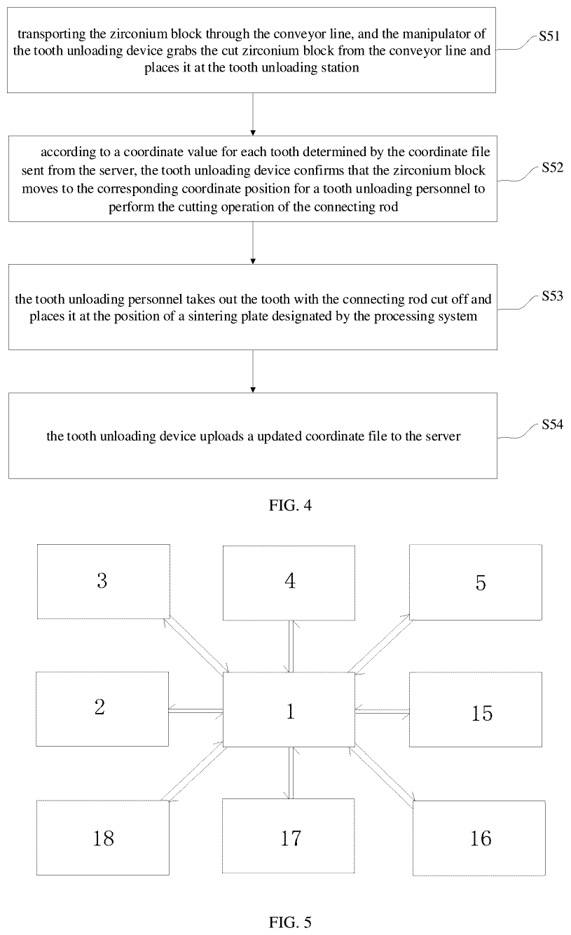

In the above Step S5, as shown in FIG. 4, the steps of transporting a cut zirconium block to a tooth unloading station, the tooth unloading device moving, according to the coordinate file, the zirconium block to a corresponding coordinate position for cutting operation of a connecting rod, taking out a cut tooth to a sintering device, and the tooth unloading device uploading the coordinate file to the server include the follows.
Step S51: transporting the zirconium block through the conveyor line, and the manipulator of the tooth unloading device grabs the cut zirconium block from the conveyor line and places it at the tooth unloading station.
Step S52: according to a coordinate value for each tooth determined by the coordinate file sent from the server, the tooth unloading device confirms that the zirconium block moves to the corresponding coordinate position for a tooth unloading personnel to perform the cutting operation of the connecting rod.

Specifically, based on the coordinate value for each tooth determined by the typesetting personnel and generated by the server, the tooth unloading device confirms the corresponding coordinate position for the zirconium block to be moved to for the tooth unloading to operate. At this point, the corresponding tooth on the zirconium block is positioned beneath the tooth unloading processing hole. The tooth unloading operator then cuts off the connecting rod of the tooth from the tooth unloading processing hole.
Step S53: the tooth unloading personnel takes out the tooth with the connecting rod cut off and places it at the position of a sintering plate designated by the processing system.
Step S54: the tooth unloading device uploads an updated coordinate file to the server.

In the above Step S2, in the step of retrieving the 3D tooth model file to the 3D zirconium block graphic file for typesetting, and generating the tool path file, the zirconium block is information-bound to the 3D zirconium block graphic file through a QR code.

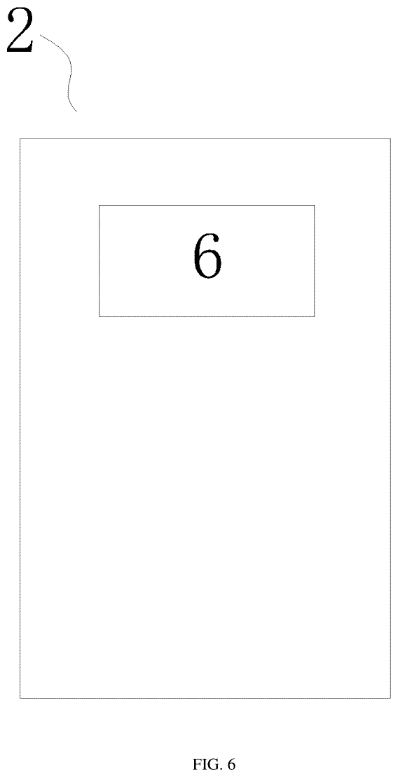

In addition, as shown in FIG. 5, the present disclosure also provides a flexible intelligent manufacturing and production system for a customized denture, wherein the flexible intelligent manufacturing and production system for the customized denture comprises a server, a processing system, a cutting device, a tooth unloading device and a sintering device; the processing system, the cutting device, the tooth unloading device and the sintering device all have the function of receiving/sending commands, and are all connected to the server signal; the processing system is used for processing a 3D tooth model file and a 3D zirconium block graphic file, and generating a file; the server is used for receiving/sending the file; the cutting device is used for receiving a tool path file and cutting a zirconium block according to the tool path file; the tooth unloading device is used for receiving/sending a coordinate file and cutting a connecting rod according to the coordinate file; and sintering device is used for sintering a denture.

In some embodiments, the flexible intelligent manufacturing and production system for the customized denture further comprises a glazing and sintering device, a sandblasting device, a polishing device and a quality inspection and packaging device; the glazing and sintering device, the sandblasting device, the polishing device and the quality inspection and packaging device all have the function of receiving/sending commands, and are all connected to the server signal; the glazing and sintering device is used for glazing and sintering a denture; the sandblasting device is used for sandblasting dentures, the polishing device is used for polishing the denture; and the quality inspection and packaging device is used for quality inspection and packaging of the denture.

When in use, the operator designs the 3D tooth model file through the processing system 2 and uploads the 3D tooth model file to the designated folder of the server 1. The typesetting personnel retrieves the 3D zirconium block graphic file in the processing system 2 and retrieves the 3D tooth model file from the server 1. The 3D tooth model file is retrieved to the 3D zirconium block graphic file for typesetting, and the processing system 2 generates a tool path file. Each tooth is numbered according to the typesetting graphic file, and the coordinates of each tooth is set and a coordinate file is generated. The tool path file and coordinate file are uploaded to the server 1, the server 1 transmits the coordinate file to the tooth unloading device 4, and transmits the tool path file to the cutting device 3, and the cutting device 3 grabs the corresponding zirconium block for cutting. The cut zirconium block is transported to the tooth unloading station 11. The tooth unloading device 4 moves the zirconium block to the corresponding coordinate position according to the coordinate file, so that the operator can perform cutting operation of a connecting rod, and take out the cut teeth to the sintering device 5. The tooth unloading device 4 uploads the coordinate file to the server 1. The server will record the coordinates of the teeth sintered in the sintering device and transfer the coordinates to the next station for positioning operation. The sintering device sinters the teeth, and the subsequent operations of the glazing and sintering device, sandblasting device, polishing device, and quality inspection and packaging device all perform real-time positioning and tracking of the coordinates of the teeth at each station in the above method. Therefore, the flexible intelligent manufacturing and production system for the customized denture can confirm the exact location of each tooth at a specific station. The processing system 2 tracks the progress to ensure that every station in the entire production process knows the relevant information for each tooth at their station, thereby improving production efficiency, reducing the phenomenon of constantly searching for teeth during the production process, and avoiding the phenomenon of errors, omissions, and losses that occur during the searching process. Therefore, the described flexible intelligent manufacturing and production system for the customized denture has the advantages of improving production efficiency and locating the position of each tooth.

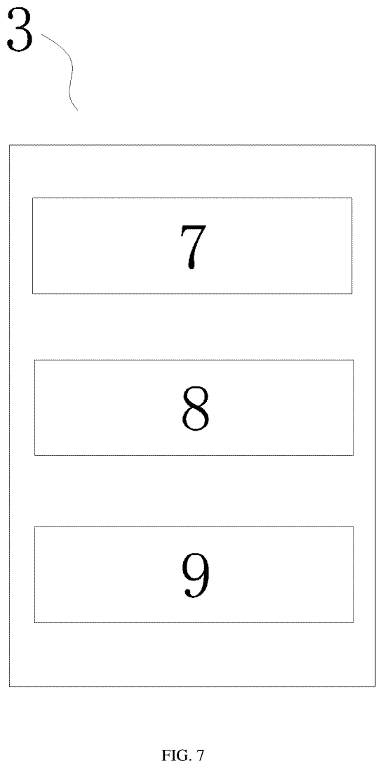

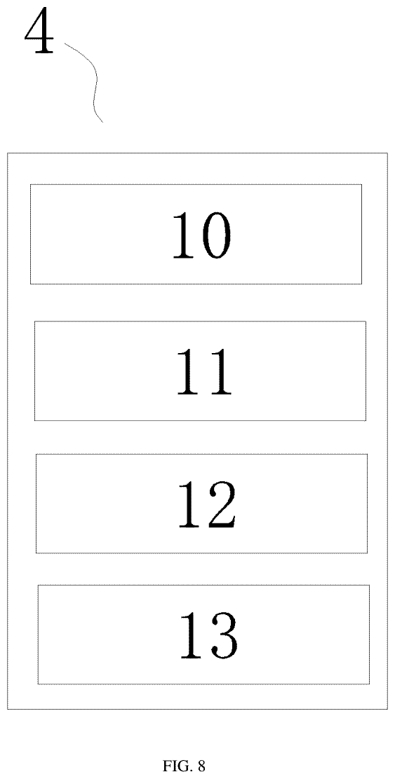

As shown in FIG. 6, in some embodiments, the processing system 2 includes a computer 6, a CAD design software is installed in the computer 6, and the 3D tooth model file designed based on the CAD design software is uploaded to a designated folder on the server 1. As shown in FIG. 7, in some embodiments, the cutting device 3 includes a first manipulator 7, a denture engraving machine 8 and a conveyor belt 9; the denture engraving machine 8 is used for engraving and cutting a tooth in the zirconium block; the first manipulator 7 is used for grabbing the corresponding zirconium block to the denture engraving machine 8 for engraving and cutting according to the server's 1 command, and placing the engraved and cut zirconium block on the conveyor belt 9; and the conveyor belt 9 is used for transporting the zirconium block to the tooth unloading device 4.

As shown in FIG. 8, in some embodiments, the tooth unloading device 4 includes a second manipulator 10, a tooth unloading station 11, a tooth unloading processing hole 12 and a cutting module 13; the second manipulator 10 is used for grabbing the zirconium block on the conveyor belt 9 to the tooth unloading station 11; the tooth unloading station 11 is used for moving the zirconium block to the corresponding coordinate position according to a coordinate value of each tooth, so that the corresponding tooth in the zirconium block is positioned beneath the tooth unloading processing hole 12; and the cutting module 13 is used by an operator to cut the corresponding tooth from the tooth unloading processing hole 12.

It should be noted that this embodiment is a system embodiment corresponding to the aforementioned method embodiment, and this embodiment can be implemented in coordination with the aforementioned method embodiment. The relevant technical details mentioned in the aforementioned method embodiment are still valid in this embodiment, and to avoid repetition, they will not be reiterated here. Correspondingly, the relevant technical details mentioned in this embodiment can also be applied to the aforementioned method embodiment.

Although the embodiments of the present disclosure are described with practical solutions, they do not constitute limitations on the meaning of the present disclosure. For those skilled in the art, modifications to the embodiments and combinations with other solutions based on this specification are obvious.

What is claimed is:

1. A flexible intelligent manufacturing and production system for a customized denture, comprising a server, a processing system, a cutting device, a tooth unloading device and a sintering device; wherein the processing system, the cutting device, the tooth unloading device and the sintering device all have a function of receiving/sending commands, and are all connected to the server signal; the processing system is used for processing a three-dimensionality (3D) tooth model file and a 3D zirconium block graphic file, and generating a file; the server is used for receiving/sending the file; the cutting device is used for receiving a tool path file and cutting a zirconium block according to the tool path file; the tooth unloading device is used for receiving/sending a coordinate file and cutting a connecting rod according to the coordinate file; and the sintering device is used for sintering a denture.

2. The flexible intelligent manufacturing and production system for the customized denture according to claim 1, further comprising a glazing and sintering device, a sandblasting device, a polishing device and a quality inspection and packaging device; wherein the glazing and sintering device, the sandblasting device, the polishing device and the quality inspection and packaging device all have the function of receiving/sending commands, and are all connected to the server signal; the glazing and sintering device is used for glazing and sintering a denture; the sandblasting device is used for sandblasting dentures, the polishing device is used for polishing the denture; and the quality inspection and packaging device is used for quality inspection and packaging of the denture.

3. The flexible intelligent manufacturing and production system for the customized denture according to claim 1, wherein the processing system comprises a computer, a computer-aided design (CAD) design software is installed in the computer, and the 3D tooth model file designed based on the CAD design software is uploaded to a designated folder on the server.

4. The flexible intelligent manufacturing and production system for the customized denture according to claim 3, wherein the cutting device comprises a first manipulator, a denture engraving machine and a conveyor belt; the denture engraving machine is used for engraving and cutting a tooth in the zirconium block; the first manipulator is used for grabbing the corresponding zirconium block to the denture engraving machine for engraving and cutting according to the server's command, and placing the engraved and cut zirconium block on the conveyor belt; and the conveyor belt is used for transporting the zirconium block to the tooth unloading device.

5. The flexible intelligent manufacturing and production system for the customized denture according to claim 4, wherein the tooth unloading device comprises a second manipulator, a tooth unloading station, a tooth unloading processing hole and a cutting module; the second manipulator is used for grabbing the zirconium block on the conveyor belt to the tooth unloading station; the tooth unloading station is used for moving the zirconium block to the corresponding coordinate position according to a coordinate value of each tooth, so that the corresponding tooth in the zirconium block is positioned beneath the tooth unloading processing hole; and the cutting module is used by an operator to cut the corresponding tooth from the tooth unloading processing hole.
